(19) United States
(12) Patent Application Publication
(10) Pub. No.: US 2021/0113815 A1
(43) Pub. Date: Apr. 22, 2021

(54) CATHETER SECUREMENT DEVICE

(71) Applicants: Naimul KARIM, Saint Paul, MN (US); 3M INNOVATIVE PROPERTIES COMPANY, SAINT PAUL, MN (US)

(72) Inventors: NAIMUL KARIM, MAPLEWOOD, MN (US); JENNIFER N. HANSON, WOODBURY, MN (US); JIA HU, MOUNDS VIEW, MN (US); CHAODI LI, WOODBURY, MN (US); THOMAS G. SKULLEY, SAINT PAUL, MN (US); SHILPI K. SANGHI, JERSEY CITY, NJ (US); PETER M. EISENBERG, MINNEAPOLIS, MN (US)

(73) Assignee: 3M INNOVATIVE PROPERTIES COMPANY, SAINT PAUL, MN (US)

(21) Appl. No.: 16/975,158

(22) PCT Filed: Feb. 18, 2019
(86) PCT No.: PCT/IB2019/051299

§ 371 Date: Aug. 24, 2020

Related U.S. Application Data
(60) Provisional application No. 62/635,640, filed on Feb. 27, 2018.

(51) Int. Cl. A61M 25/02

(52) U.S. Cl. CPC A61M 25/02; A61M 2025/024

(57) ABSTRACT

A securement device for securing a medical article, comprising abase having a longitudinal axis, a top face and a bottom face, where the longitudinal axis defines a longitudinal direction, a cuff disposed on the top face of the base and positioned along the longitudinal axis, the cuff comprising an entry gap in at least a portion of the cuff and defining an interior aperture, and a bar disposed on the top face of the base, the bar positioned along the longitudinal axis, where the bar is moveable between an open position and a closed position and where the bar cooperates with the cuff in the closed position to secure the medical article.

### BACKGROUND

Various medical treatments often require the use of medical articles and tubing. In many cases the devices or tubing must be secured to a patient's body. For example, it can be necessary to introduce fluids and liquid medications directly into a blood vessel of a patient and for short term general use, a simple intravenous (“IV”) line can be placed onto a patient's arm. For longer term and more specialized needs, catheters or other devices are used, such as, for example, a Foley catheter, that may be necessary for draining urine from a patient's bladder.

Healthcare providers often secure catheters and other devices or tubing to patients during hospital stays or in-home care. Securing the devices aids in proper positioning, which prevents leaks or interruptions in medication dosing, minimizes patient discomfort, and limits tangling, catching, and dislodging of connective tubing due to patient movement. In order to keep a catheter or other medical articles or tubing properly positioned for the duration of treatment, the medical article may be secured to the patient in a variety of ways.

One common way of securing a medical article or tubing to a patient is by taping the catheter or medical line to the patient's skin. However, taping can be time consuming and labor intensive. Tape can also collect contaminants and must be frequently removed and replaced. In addition, taping is not necessarily effective in securing a medical article or catheter in place, and removal of the tape may cause undesired motion of the device or catheter. Sutures have also been used to attach a catheter to a patient. With sutures, the catheter is stitched onto the patient's skin. Sutures, however, can be a source of infection, can cause pain and inflammation, and can make it more difficult to clean around the incision site. Sutures also require time and skill to place, and can cause scarring. As a result, many practitioners are moving away from securement with tapes and sutures to securement with dedicated devices that are designed to give consistent results and minimize user error.

### SUMMARY

In one aspect, provided is a securement device for securing a medical article, comprising a base having a longitudinal axis, a top face and a bottom face, where the longitudinal axis defines a longitudinal direction, a cuff disposed on the top face of the base and positioned along the longitudinal axis, the cuff comprising an entry gap in at least a portion of the cuff and defining an interior aperture, and a bar disposed on the top face of the base, the bar positioned along the longitudinal axis, where the bar is moveable between an open position and a closed position and where the bar cooperates with the cuff in the closed position to secure the medical article.

In another aspect, provided is a securement system comprising a securement device including a base having a longitudinal axis, a top face and a bottom face, where the longitudinal axis defines a longitudinal direction, a cuff disposed on the top face of the base and positioned along the longitudinal axis, the cuff comprising an entry gap in at least a portion of the cuff and defining an interior aperture, and a bar disposed on the top face of the base, the bar positioned along the longitudinal axis, where the bar is moveable between an open position and a closed position and where the bar cooperates with the cuff in the closed position to secure the medical article, and a support device.

Features and advantages of the present disclosure will be further understood upon consideration of the detailed description as well as the appended claims.

### BRIEF DESCRIPTION OF THE DRAWINGS

FIG. 1A is a perspective view of a first embodiment of a securement device in a closed configuration.

FIG. 1B is a perspective view of a first embodiment of a securement system with the securement device in a closed configuration.

FIG. 1C is a side view of the securement system of FIG. 1B.

FIG. 2 is a side view of a second embodiment of a securement device with the securement device in a closed configuration.

FIG. 3A is a perspective view of a third embodiment of a securement device with the securement device in a closed configuration and an unsecured medical article positioned on the bar of the securement device.

FIG. 3B is a perspective view of the securement device of FIG. 3A with securement device in an open configuration and the medical article entering the cuff opening.

FIG. 3C is a side view of the securement device of FIGS. 3A and 3B with the securement device in a closed configuration and securing the medical article.

FIG. 4 is a perspective view of a fourth embodiment of a securement system with the securement device in a closed configuration.

FIG. 5 is a perspective view of a fifth embodiment of a securement device in a closed configuration and a medical device.

FIG. 6A is a perspective view of a sixth embodiment of a securement device in a closed configuration and a medical device.

FIG. 6B is a perspective view of a sixth embodiment of a securement device in an open configuration.

FIG. 7A is a perspective view of a seventh embodiment of a securement device in an open configuration.

FIG. 7B is a perspective view of a seventh embodiment of a securement device in a closed configuration.

Repeated use of reference characters in the specification and drawings is intended to represent the same or analogous features or elements of the disclosure. It should be understood that numerous other modifications and embodiments can be devised by those skilled in the art, which fall within the scope and spirit of the principles of the disclosure. The figures may not be drawn to scale.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the term “coupled” and variations thereof are used broadly and encompass both direct and indirect couplings. It is to be understood that other embodiments may be utilized, and structural or logical changes may be made without departing from the scope of the present disclosure. Furthermore, terms such as “side,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

The present disclosure generally relates to medical article securement devices and systems and methods for safely and reliably securing a medical article, such as a catheter system, upon a desired location of a patient's body. The medical article securement systems can be universal to accommodate and reliably secure a large variety of medical articles or class of medical articles (e.g., Foley catheters, peripherally inserted central catheters) and can be particularly useful for securing medical articles that need to be secured to a patient over a prolonged period of time, such as weeks or months.

Examples of medical articles that can be employed with the medical article securement devices and systems of the present disclosure include, but are not limited to, connector fittings, catheter systems (e.g., catheters, catheter hubs, catheter adaptors), fluid supply lines, other similar articles, or combinations thereof. Examples of catheter systems can include, but are not limited to, Foley catheters, intravenous (“IV”) catheters, central venous catheters (“CVCs”), peripherally inserted central catheters (“PICCs”), arterial catheters, and dialysis catheters.

The phrase “interior aperture circumference” as used herein refers to the circumference of the interior aperture 143 of cuff 140, calculated according to the equation c=π·d, where “d” is the length of the inner diameter of the cuff 140, i.e., the diameter of the interior aperture 143, and circumference “c” includes the length of entry gap 142.

The phrase “entirely contiguous” as used herein means that the surface area is of substantially continuous material construction and is substantially free of large voids, gaps, or perforations in the material construction. On a small scale, a material may still be entirely contiguous even if there are gaps in the material. For example, films, nonwovens, paper, and fabrics could all be formed in a way such that it is entirely contiguous.

The phrase “highly moisture vapor permeable” means that the material transmits moisture vapor at a rate similar to or greater than human skin. For example, using the inverted cup method as described in U.S. Pat. No. 4,595,001, highly moisture vapor permeable means having a rate of at least 300 g/m²/24 hours at 37° C./100-10% relative humidity.

The phrase “liquid water impermeable” means that if liquid water is put in direct contact with one surface of the material then, under normal atmospheric pressure, it is not readily transported to the opposite surface of the material.

The term “longitudinal” is used to refer to a direction or axis that is generally perpendicular to the direction in which the medical article extends when it is secured in the securement device and generally perpendicular to the overall direction of fluid flow, e.g., along a catheter line.

As used herein, “a,” “an,” “the,” “at least one,” and “one or more” are used interchangeably.

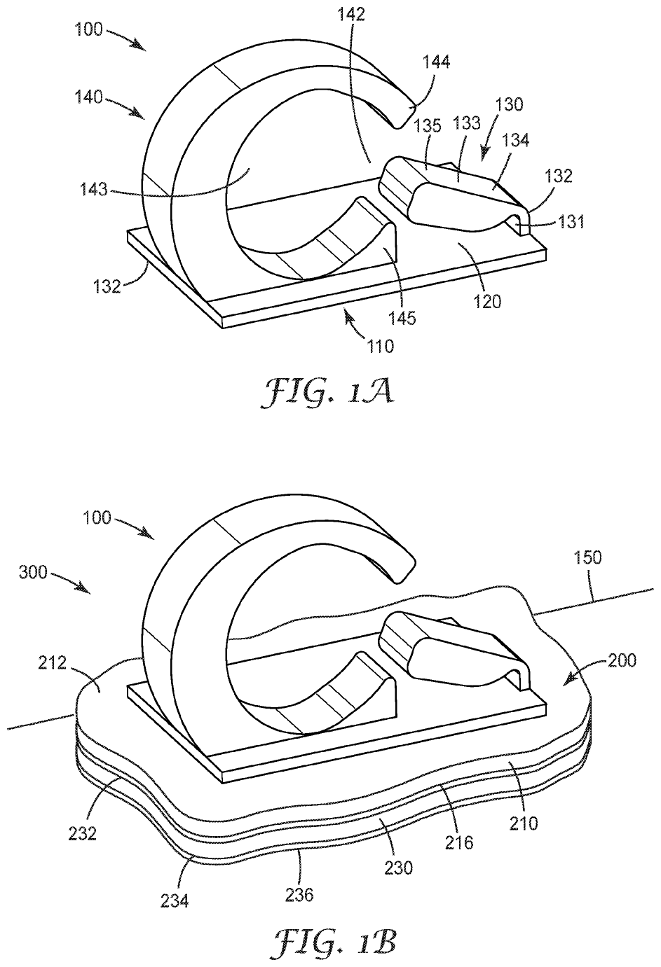

FIG. 1A a perspective view of a first embodiment of a securement device 100 in a closed configuration. FIG. 1B a perspective view of a first embodiment of a securement system 300 with the securement device 100 in a closed configuration.

The securement device 100 comprises a base 110 having a longitudinal axis 150, a top face 120 and a bottom face 121. The longitudinal axis 150 defines a longitudinal direction. A base 110 of the present disclosure can be constructed of either rigid or flexible materials. The base 110 can have any suitable shape or footprint. In some embodiments, the base 110 may have a rectangular shape.

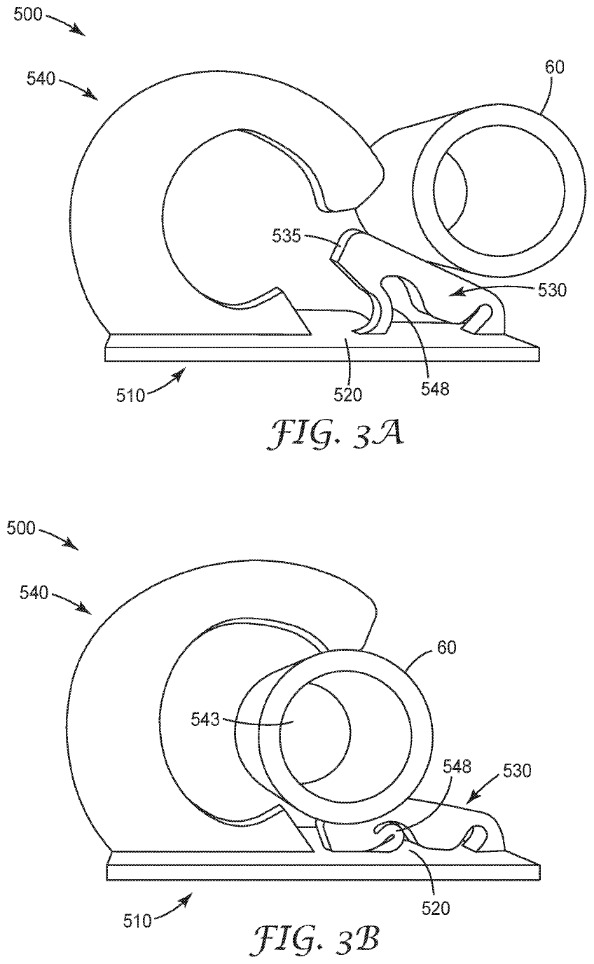

The securement device 100 further comprises a cuff 140 disposed on the top face 120 of the base 110 and positioned along the longitudinal axis, the cuff 140 defining an interior aperture 143. The cuff 140 further comprises an entry gap 142. The cuff 140 may be configured to receive and resiliently retain a generally cylindrical object such as, for example, medical tubing 60 (shown in FIGS. 3A-3C).

In some embodiments and as shown in FIGS. 1A and 1B, the entry gap 142 in the cuff 140 may be smaller than the interior aperture 143 diameter defined by the cuff 140 such that a generally cylindrical object e.g., tubing, can be firmly secured within the interior aperture 143 of the cuff 140 via press-fit or snap-fit insertion. The cuff 140 may be configured such that the maximum width of a generally cylindrical object such as, for example, medical tubing 60, is greater than the maximum width of the entry gap 142 when the cuff 140 is in a non-deflected or closed state. As a result, cuff edges 144, 145 may spread apart or deflect outward to accommodate the generally cylindrical object when it is inserted into entry gap 142 of cuff 140. If desired, cuff edges 144, 145 may be fabricated to angle inward toward entry gap 142 and interior aperture 143 to facilitate the insertion of a generally cylindrical object and the corresponding deflection of cuff edges 144, 145.

In some embodiments, cuff 140 may be sufficiently rigid such that cuff edges 144, 145 do not spread apart or deflect when a generally cylindrical object such as a medical article is pressed into cuff 140 through the entry gap 142. In these embodiments, the medical article may deform to allow entry through the entry gap 142 and into the interior aperture 143. In some embodiments, the cuff edges 144, 145 resiliently spread apart or deflect, while at the same time, the medical article may deform, both working cooperatively to allow entry of the medical article through the entry gap 142 and into the interior aperture 143. In some embodiments, the size of the entry gap 142 is comparable to the diameter of a medical article, e.g., catheter tube, to be secured. In some embodiments, the length of the entry gap 142 is less than 50%, less than 45%, less than 40%, less than 35%, less than 30%, less than 25%, or less than 20% of the interior aperture circumference. In some embodiments, the length of the entry gap 142 is at least 5%, at least 10%, at least 15%, at least 20%, at least 25%, at least 30%, or at least 35% of the interior aperture circumference. In some embodiments, the length of the entry gap 142 is 5% to 45%, 5% to 40%, or 5% to 35% of the interior aperture circumference. In some preferred embodiments, the length of the entry gap 142 may be 10% to 30% (e.g., 20%) of the interior aperture circumference.

In some embodiments, cuff 140 is generally annular in shape. In some embodiments, the interior aperture 143 of the cuff 140 is generally circular in shape, but it may be another shape such as, for example, elliptical, tear-drop shaped, square, rectangular, or triangular. The entry gap 142 may be positioned above the base 110 at or near the center of the cuff 140, as shown in FIGS. 1A and 1B. In some embodiments, the entry gap 142 may be positioned such that it directly abuts the top face 120 of the base 110.

In some embodiments, the securement device 100 further comprises a bar 130. In the exemplary embodiment shown in FIGS. 1A and 1B, the bar 130 comprises a post 131 extending from the top face 120 of the base 110, though more than one post 131 may be suitable in some embodiments. The bar 130 further comprises a shaft 133 having a first shaft end 134 and a second shaft end 135. The first shaft end 134 is coupled to the post 131 via a flexible joint 132. Exemplary flexible joints 132 can include hinges and living hinges. In some embodiments the flexible joint 132 need not be a hinge, but may be constructed of a flexible material. In some embodiments, flexible joint 132 can be flexible, particularly, relative to the post 131 and shaft 130, and the post 131 and shaft 130 can be rigid relative to the flexible joint 132, even when the post 131, shaft 130, and flexible joint 132 are made of the same material. In some embodiments, post 131 and shaft 130 may be sufficiently rigid such that post 131 and shaft 133 do not deform significantly when shaft 133 is pressed toward the top face 120 of the base 110.

The bar 130 is moveable between a closed position (shown in FIGS. 1A and 1B) and an open position. When the bar 130 is in the closed position, the second shaft end 135 is positioned next to the entry gap 142 of the cuff 140, such that second shaft end 135 of the bar 130 acts to block the entry gap 142, i.e., the bar 130 cooperates with the cuff entry gap 142 in the closed position to form an obstacle to a medical article from leaving the interior aperture 143 after it has been positioned therein. In some embodiments, and as shown in FIGS. 1A and 1B, when in the closed position the second shaft end 135 of the bar 130 does not extend across the entire length of the cuff entry gap 142, i.e., the second shaft end 135 of the bar 130 does not extend from cuff edge 144 to cuff edge 145. In some embodiments, when in the closed position the second shaft end 135 of the bar 130 may block 100%, at least 90%, at least 80%, at least 70%, at least 60%, at least 50%, at least 40%, at least 30%, or at least 20% of the entry gap 142. In some embodiments, when in the closed position, the second shaft end 135 of the bar 130 may block up to 10%, up to 20%, up to 30%, up to 40%, up to 50%, up to 60%, up to 70%, up to 80%, up to 90%, or up to 100% of the entry gap 142. In some embodiments, when in the closed position, the second shaft end 135 of the bar 130 may block 1% to 100%, 10% to 96%, 20% to 92%, 30% to 88%, 40% to 84%, or 50% to 80% of the entry gap 142. In some preferred embodiments, when in the closed position, the second shaft end 135 of the bar 130 may block 60% to 90% (e.g., 75%) of the entry gap 142.

When the bar 130 is in the open position, which may be achieved, for example, by applying a downward pressure to the shaft 133, i.e., pressing the shaft 133 toward the top face 120 of the base 110, the second shaft end 135 is positioned such that second shaft end 135 of the bar 130 does not block the entry gap 142 or blocks the entry gap 142 less than when the bar is in the closed position, thus allowing entry of a medical article through the entry gap 142 and into the interior aperture 143. Once the medical article has entered the interior aperture 143, downward pressure on the shaft 133 may be released so that the bar 130 returns to the closed position and the bar 130 helps to prevent the medical article from leaving the interior aperture 143. In the embodiment shown in FIGS. 1A and 1B, the second shaft end 135 partially blocks the entry gap 142, is not coupled to any other portion of the securement device 100, e.g., the base 110 or a cuff edges 144,145, and does not extend into the interior aperture 143.

In some embodiments, the bar 130 and the cuff 140 are each independently coupled to the base 110. Exemplary means of coupling the bar 130 and the cuff 140 to the base 110 and in some embodiments coupling other elements of the securement device 100 to each other, e.g., coupling second shaft end 135 to compressible segment 148, coupling compressible segment 148 to cuff edge 145, include, but are not limited to, one or more of adhesives, cohesives, magnets, welding (e.g., sonic (e.g., ultrasonic) welding), a thermal bonding or heat sealing technique (e.g., heat and/or pressure applied to one or both of the components to be coupled), other suitable coupling means, or combinations thereof. Additionally, coupling can be accomplished by forming elements of the securement device 100, e.g., the base 110, the bar 130, the cuff 140, integrally from a unitary material to form a unitary piece by methods known in the art, such as, for example, injection molding, extrusion molding, milling, 3D printing, and combinations thereof. In some embodiments, elements of the securement device 100 are independently formed from differing materials. In some embodiments, elements of the securement device 100 are independently formed from the same material. The robustness of the materials used to make the securement device 100 of the present disclosure can offer facile, reliable, repeatable, and secure coupling and decoupling of a medical article to securement device 100.

Securement devices of the present disclosure can be constructed of any suitable material that allows both appropriate flexibility and rigidity. In some embodiments, the securement devices of the present disclosure may be constructed of polymeric or elastomeric materials. In some embodiments, the securement devices of the present disclosure may be constructed of metals, plastics, or composites. Exemplary materials include ABS plastic, polypropylene, polycarbonate, polyethylene, polyvinylchloride, acrylonitrile butadiene styrene copolymer, nylon, polyolefins, acrylics, polyesters, silicones, thermoplastic polyurethanes, thermoplastic elastomers, and the like.

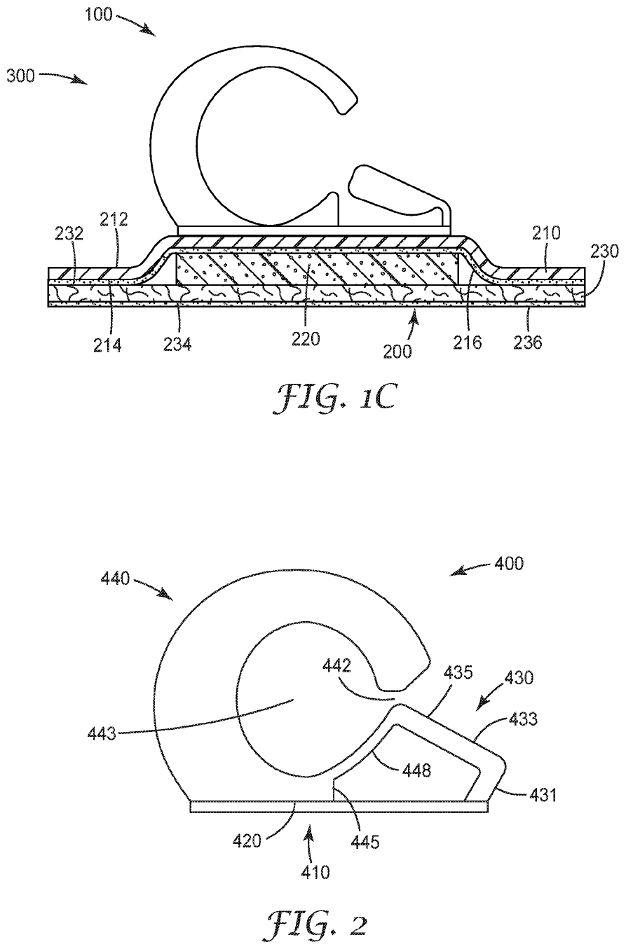

In some embodiments, and as shown in a second embodiment of the securement device 400 in FIG. 2, the bar 430 may include a second shaft end 435 further comprising a compressible segment 448 coupled to cuff edge 445, i.e., the lower cuff edge. Referring to FIG. 2, the compressible segment 448, shown fully extended, can extend from the second shaft end 435 to the cuff edge 445, blocking a portion of the entry gap 142 when the securement device 400 bar 430 is in the closed position. When the bar 430 is moved to the open position, as described above, the compressible segment 448 may deform or partially deform and allow a medical article to pass through the entry gap 442 into the interior aperture 443 of the cuff 440. Once the medical article has entered the interior aperture 443, the bar 430 returns to the closed position with the bar 430 including the fully extended compressible segment 448 cooperating to prevent the medical article from leaving the interior aperture 443.

In some embodiments, and as shown in a third embodiment of the securement device 500 in FIGS. 3A-3C, the bar 530 may include a second shaft end 535 further comprising a compressible segment 548 coupled to the top face 520 of the base 510. FIG. 3A shows the securement device 500 in a closed position, the compressible segment 548 fully extended, with an unsecured medical article 60. FIG. 3B shows the securement device 500 in the open position, the bar 530 having been moved toward the top face 520 of the base 510 and the compressible segment 548 fully compressed to allow the medical device 60 to pass through the entry gap 542 and into the interior aperture 543. FIG. 3C shows the securement device 500 returned to a closed position, the compressible segment 548 fully extended, and the secured medical article 60.

The compressible segment 448, 548 is desirably constructed of a flexible, compressible material such that it readily deforms when shaft 433, 533 is pressed toward the top face 420, 520 of the base 410, 510. In some embodiments, compressible segment 448, 548 can be flexible, particularly, relative to the post 431, 531 and shaft 430, 530, and the post 431, 531 and shaft 430, 530 can be relatively rigid, relative to the compressible segment 448, 548 even when the post 431, 531, shaft 430, 530, and compressible segment 448, 548 are made of the same material.

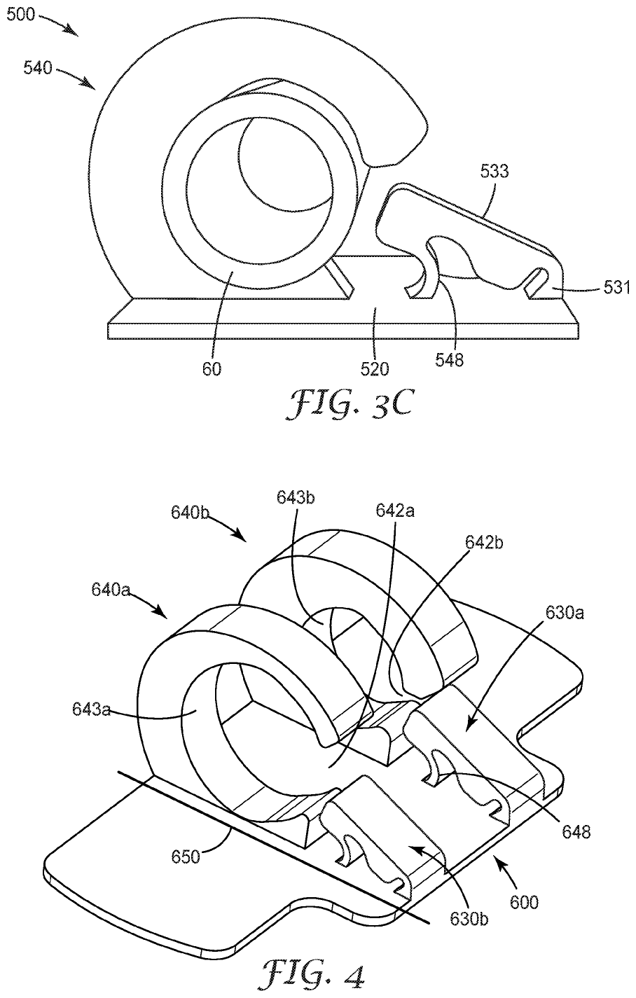

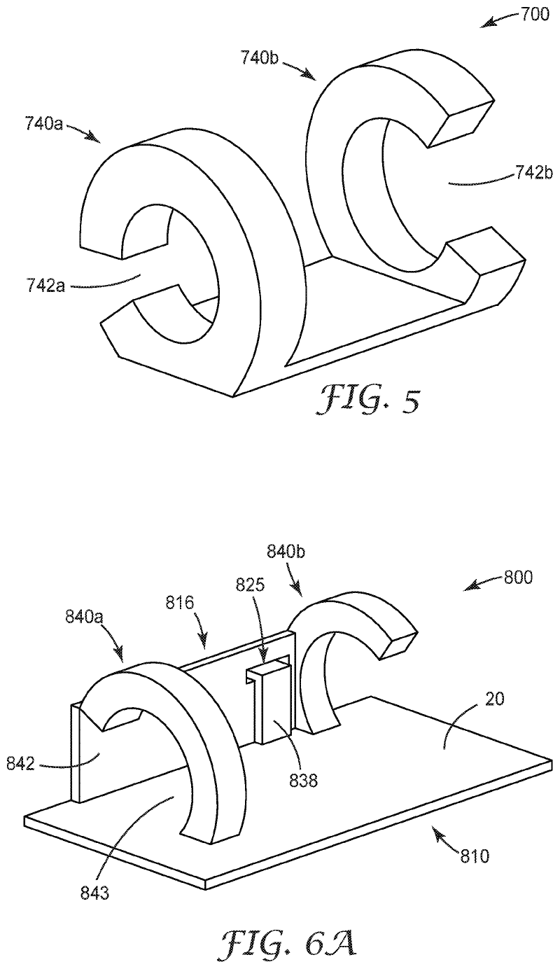

In some embodiments, and as shown in a fourth embodiment of the securement device 600, the securement device 100, 400, 500, 600 may include more than one cuff 640. In some embodiments, a first cuff 640a and a second cuff 640b are spaced a distance apart along a line perpendicular to longitudinal axis 650, as shown in FIG. 4, with entry gaps 642a, 642b aligned on the same side of the securement device 600. In FIG. 4, the cuffs 640a, 640b have different sized interior apertures 643a, 643b and the same type of compressible segment 648, but this need not be the case, and the skilled practitioner will recognize that many combinations of cuff 640 size, interior aperture 643 size, and type of compressible segment 648 are possible (see, e.g., compressible segments in FIGS. 2 and 3A-3C). For example, in some embodiments, the cuffs 640a, 640b could be of different sizes, e.g., the first cuff 640a could be larger than the second cuff 640b; in some embodiments, the first cuff 640a may be paired with a bar 630a including a first type of compressible segment 648, and the second cuff 640b may be paired with a bar 630b including a second type of compressible segment 648; in some embodiments, the first cuff 640a may be paired with a bar 630a including a first type of compressible segment 648, and the second cuff 640b may be paired with a bar 630b that does not include a compressible segment 648 (see, e.g., bar 130 in FIG. 1); or in some embodiments, the first cuff 640a and the second cuff 640b may both be paired with a bar 630a, 630b that does not include a compressible segment 648. In some embodiments, and as shown in a fifth embodiment of the securement device 700 (bar 730 not shown), the entry gaps 742a, 742b of the first cuff 740a and second cuff 740b may be positioned on opposite sides of their respective cuffs 740a, 740b with respect to each other, as shown in FIG. 5. Though not shown in FIG. 5, it should be appreciated that at least one cuff 740a, 740b is paired with a bar 730, the bar 730 which could be of the types shown in FIGS. 1, 2, and 3A-3C.

The securement device 100 can be applied to a support device 200 to form a securement system 300. The support device 200 can comprise one or more materials that aid in providing a connection of the securement device 100 to the surface to which the securement device 100 is connected. For example, the support device 200 can be a single layer of material with the securement device 100 on one side, and adhesive on the opposite side. Suitable materials could include films, paper, woven, knitted, or nonwoven fabrics. In one embodiment, the support device 200 includes elements that aid in cushioning between the securement device 100 and the surface to which the securement device 100 is connected.

On embodiment of a support device 200 that may be suitable for use with the securement device 100 is described in U.S. Patent Publication No. 2016-0166422 (Karim et al.) incorporated herein by reference. In one embodiment, as shown in FIGS. 1B and 1C, the support device 200 comprises a backing layer 210, a base layer 230, and a cushioning element 220 positioned between the backing layer 210 and base layer 230.

Referring to FIGS. 1B and 1C, the backing layer 210 comprises a first surface 212 and second surface 214, opposite the first surface. The first surface 212 of the backing layer 210 typically faces away from the surface to which the support device 200 attaches. Also, the first surface 212 contacts the bottom face 121 of securement device 100. The backing layer 210 is highly moisture vapor permeable and the first surface 212 of the backing layer 210 is liquid water impermeable.

The cushioning element 220 is placed adjacent the backing layer 210 and base layer 230. The cushioning element 220 provides comfort and helps prevent a securement device 100 from irritating or damaging the underlying surface. Therefore, typically the cushioning element 220 is deformable and compressible. Suitable materials for the cushioning element 220 can include a foam, a sponge, a gel, a hydrocolloid, a nonwoven material, a woven material, a knitted material, and combinations thereof. In one embodiment the cushioning element 220 is nonabsorbent.

In one embodiment, the cushioning element 220 has a thickness substantially greater than a thickness of the backing layer 210 and base layer 230. In one embodiment, the cushioning element 220 is nonabsorbent. In one embodiment, the cushioning element 220 is highly moisture vapor permeable. In one embodiment, the cushioning element 220 can distribute pressure.

The base layer 230 comprises a first surface 232 adjacent the cushioning element 220 and a second surface 234, opposite the first surface 232 of the cushioning element 220. The second surface 234 of the base layer 230 comprises an adhesive 236. In one embodiment, substantially the entire second surface 234 comprises the adhesive 236. In one embodiment, the adhesive 236 is on a portion of the second surface 234. For example, the adhesive 236 may be pattern coated to the second surface 234 of the base layer 230.

The base layer 230 is entirely contiguous. In one embodiment, the base layer 230 is of continuous and uniform material construction. In one embodiment, the base layer 230 is of substantial uniform thickness across the entire base layer 230. The base layer 230 is highly moisture vapor permeable. In one embodiment, the base layer 230 is liquid water impermeable.

The base layer 230 and backing layer 210 can each comprise one or more layers of materials. In one embodiment, the base layer 230 and backing layer 210 are of substantially the same area. In one embodiment the base layer 230 and backing layer 210 have a larger surface area than the surface area of the cushioning element 220.

To contain the cushioning element 220, the base layer 230 and backing layer 210 connect entirely around the cushioning element 220. In one embodiment, base layer 230, backing layer 210 or both the base layer 230 and backing layer 210 comprise a securing adhesive 216 for connecting the base layer 230 and backing layer 210.

In the exemplified construction, the cushioning element 220 is entirely contained within the structure of the backing layer 210 and base layer 230. The base layer 230 includes an adhesive 236 for securing the support device 200 to a surface, such as skin. The backing layer 210 includes a first surface 212 that is liquid water impermeable, which allows for easy cleaning of the support device 200. Overall, in one embodiment, the backing layer 210, cushioning element 220, and base layer 230 are highly moisture vapor permeable, making the support device 200 well suited for application to skin. Distinguishing from a wound dressing, the support device 200 includes the base layer 230 that is entirely contiguous, which provides significant containment of the cushioning element 220 from liquid.

In one embodiment, the support device 200 can be applied to skin to protect the underlying skin from contact with hard, abrading, or irritating surfaces. In one embodiment, the support device 200 can be applied to skin to protect the skin from applied pressure, which may result in a pressure ulcer on the skin. With the liquid water impermeable backing layer 210, the support device 200 can be easily cleaned.

The support device 200 is well suited for application and adhering to skin and can be cleaned without the liquid water penetrating into the cushioning element 220. Limiting absorption of liquid into the cushioning element 220 increases the time that the support device 200 can be worn on the skin without a need for removal and changing.

The securement device 100, can be applied to the first surface 212 of the backing layer 210, adjacent the cushioning element 220. The securement device 100 can be permanently or removably secured to the first surface 212. For example, tape, hook/loop, or adhesives can be used to secure the device 100 to the support device 200. In one embodiment, a securement device 100 such as shown in FIG. 1A, is permanently secured to the first surface 212 of the backing layer 210, and the securement device 100 is used to secure tubing or a catheter.

To use the securement device 100 to secure at least a portion of a medical article to a patient, a downward pressure is exerted on the shaft 133, i.e., the shaft 133 is pressed toward the top face 120 of the base 110 so the bar 130 does not block the entry gap 142, thus allowing at least a portion of a medical article, such as medical tubing 60, to be inserted through the entry gap 142 and into the interior aperture 143. Once the medical article has entered the interior aperture 143, downward pressure on the shaft 133 may be released so that the bar 130 to returns to the closed position and prevents the medical article from leaving the interior aperture 143 of the cuff 140, e.g. via press-fit or snap-fit insertion, and the securement system 300 is secured to the patient.

In one embodiment, the securement device 100 is connected to a support device 200 to form a securement system 300. The adhesive 236 on the second surface 234 of the base layer 230 of the support device 200 is applied to the patient to secure the securement system 300. In one embodiment, the securement system 300 is applied to the patient before the medical article 60 is inserted into the interior aperture 143. In one embodiment, the securement system is applied to the patient after the medical article 60 is inserted into the interior aperture 143.

In some embodiments, the medical article is a catheter or catheter tubing. In some embodiments, the catheter or catheter tubing is connected to a urine bag through urine bag tubing and the catheter or catheter tubing is secured within the first securing portion 130 and the urine bag tubing is secured within the second securing portion.

### Support Device Backing Layer

The backing layer 210 may include one or more layers of material such as non-woven and woven fibrous webs, knits, films, foams polymeric films and other familiar backing materials. In some embodiments, a transparent substrate is desirable to allow for viewing of the underlying skin or medical device. In one embodiment, the backing layer 210 has high moisture vapor permeability, but generally impermeable to liquid water so that microbes and other contaminants are sealed out from the area under the backing layer 210 and cannot penetrate into the cushioning element 220. One example of a suitable material is a high moisture vapor permeable film such as described in U.S. Pat. Nos. 3,645,835 and 4,595,001, the disclosures of which are herein incorporated by reference in their entireties. In high moisture vapor permeable film/adhesive composites, the composite should transmit moisture vapor at a rate equal to or greater than human skin such as, for example, at a rate of at least 300 g/m²/24 hrs at 37° C./100-10% RH, or at least 700 g/m²/24 hrs at 37° C./100-10% RH, or at least 2000 g/m²/24 hrs at 37° C./100-10% RH using the inverted cup method as described in U.S. Pat. No. 4,595,001. In one embodiment, the substrate is an elastomeric polyurethane, polyester, or polyether block amide film. These films combine the desirable properties of resiliency, elasticity, high moisture vapor permeability, and transparency. A description of this characteristic of backing layers can be found in issued U.S. Pat. Nos. 5,088,483 and 5,160,315, the disclosures of which are hereby incorporated by reference in their entireties.

### Support Device Base Layer

The base layer 230 provides a surface to which the adhesive 236 is applied to and also provides a surface that contains the cushioning element. In one embodiment, the base layer can be of a construction substantially as described above for the backing layer 210, and therefore includes non-woven and woven fibrous webs, knits, films, foams polymeric films and other familiar backing materials. It may be desirable that the base layer be kept relatively thin to, e.g., improve conformability. For example, the base layer may be formed of polymeric films with a thickness of 200 micrometers or less, or 100 micrometers or less, potentially 50 micrometers or less, or even 25 micrometers or less. The base layer can also be comprised of a combination of two or more materials, such as, for example, two films (coextruded), film and fabric (woven, knitted, nonwoven). In one embodiment, the base layer 230 has high moisture vapor permeability. In one embodiment, to limit introduction of liquid into the cushioning element 220, the base layer 230 is impermeable to liquid water.

### Support Device Adhesive

Suitable adhesive for use on the second surface 234 of the base layer 230 for securing the support device 200 to a surface include adhesive that provides acceptable adhesion to skin and is acceptable for use on skin (e.g., the adhesive should preferably be non-irritating and non-sensitizing). Suitable adhesives are pressure sensitive and in certain embodiments have a relatively high moisture vapor transmission rate to allow for moisture evaporation. Suitable pressure sensitive adhesives include those based on acrylates, urethane, hydrogels, hydrocolloids, block copolymers, silicones, rubber based adhesives (including natural rubber, polyisoprene, polyisobutylene, butyl rubber etc.) as well as combinations of these adhesives. The adhesive component may contain tackifiers, plasticizers, rheology modifiers as well as active components including, for example, an antimicrobial agent.

The pressure sensitive adhesives that may be used may include adhesives that are typically applied to the skin such as the acrylate copolymers described in U.S. Pat. No. RE 24,906, particularly a 97:3 isooctyl acrylate:acrylamide copolymer. Another example may include a 70:15:15 isooctyl acrylate:ethyleneoxide acrylate:acrylic acid terpolymer, as described in U.S. Pat. No. 4,737,410 (Example 31). Other potentially useful adhesives are described in U.S. Pat. Nos. 3,389,827; 4,112,213; 4,310,509; and 4,323,557. Inclusion of medicaments or antimicrobial agents in the adhesive is also contemplated, as described in U.S. Pat. Nos. 4,310,509 and 4,323,557.

Silicone adhesive can also be used. Generally, silicone adhesives can provide suitable adhesion to skin while gently removing from skin. Suitable silicone adhesives are disclosed in PCT Publications WO2010/056541 and WO2010/056543, the disclosure of which are herein incorporate by reference in their entireties.

The pressure sensitive adhesives may, in some embodiments, transmit moisture vapor at a rate greater to or equal to that of human skin. While such a characteristic can be achieved through the selection of an appropriate adhesive, it is also contemplated that other methods of achieving a high relative rate of moisture vapor transmission may be used, such as pattern coating the adhesive, as described in U.S. Pat. No. 4,595,001. Other potentially suitable pressure sensitive adhesives may include blown-micro-fiber (“BMF”) adhesives such as, for example, those described in U.S. Pat. No. 6,994,904. The pressure sensitive adhesive used may also include one or more areas in which the adhesive itself includes structures such as, e.g., the microreplicated structures described in U.S. Pat. No. 6,893,655.

Issued U.S. Pat. Nos. 3,645,835 and 4,595,001, the disclosures of which are hereby incorporated by reference in their entireties, describe methods of making films and methods for testing their permeability. Preferably, the film/adhesive composite should transmit moisture vapor at a rate equal to or greater than human skin. Preferably, the adhesive coated material transmits moisture vapor at a rate of at least 300 g/m²/24 hrs at 37° C./100-10% RH; or in one embodiment at a rate of at least 700 g/m²/24 hrs at 37° C./100-10% RH; or in one embodiment at a rate of at least 2000 g/m²/24 hrs at 37° C./100-10% RH using the inverted cup method as described in U.S. Pat. No. 4,595,001.

Different portions of the support device 200 may include different adhesives, such as disclosed in U.S. Pub. No. 2015/0141949 (Decabooter et al.). For example, a portion may include an acrylate adhesive while another portion may include a silicone adhesive. In one embodiment, to prevent edge separation, adjacent the perimeter is acrylate adhesive, while near the central portion there is silicone adhesive. In one embodiment, to strongly secure with a device or tubing near the central portion there is acrylate adhesive, while near the perimeter in contact with skin is silicone adhesive.

### Optional Components

An optional release liner may be included that covers all or a portion of the adhesives to prevent contamination of the adhesives. In one embodiment, the package that contains the support device 200 may serve as a release liner.

An optional carrier may be included that covers all or a portion of the first surface 212 of the backing layer 210, providing structural support if the backing 210 is thin and highly flexible. The carrier may be removable from the first major surface 212 once the support device 200 is placed on skin. The carrier can be constructed of a variety of materials such as fabric that are woven or kitted, nonwoven material, papers, or film. In one embodiment, the carrier is along the perimeter of the first surface 212 of the backing layer 210 and is removable from the first major surface 212 similar to the carrier used in products such as 3M TEGADERM Transparent Film Dressing, available from 3M Company, St. Paul, Minn.

The present disclosure can also provide indicia for use with the securement device 100 or system 300 of the present disclosure. In some embodiments the indicia include a representation (e.g., a pictorial representation) of a medical article of interest, such that the indicia mimic the overall shape, appearance and/or configuration of the medical article to provide a visual cue for how to couple the medical article to the securement device or system. In some embodiments, the indicia include directional cues and/or a representation (e.g., a pictorial representation) of a patient's body. Directional cues, such as arrows, may indicate how the system should be oriented relative to another device, structure, or portion of a patient's body. Support devices of the present disclosure can also include such directional cues. Such indicia can enhance the usability of the systems of the present disclosure and can minimize operator errors when applying the systems to patients and coupling medical articles to the devices and systems.

The indicia can include a variety of markings, graphics, or the like, in order to represent a medical article. For example, in some embodiments, the indicia can include a two-dimensional representation of the outline, outer contours, or outer periphery of a medical article. As such, the indicia may be a simplified representation of the medical article, but it will be clear to a user how to orient the medical article relative to the system, based on the caricature or representation of the medical article provided by the indicia.

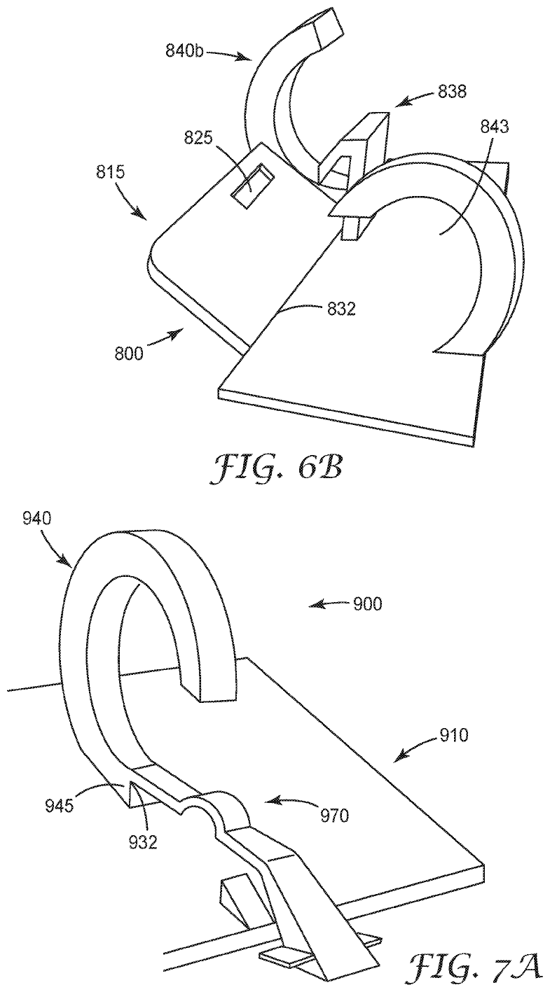

Another embodiment of a securement device 800 is shown in FIGS. 6A (closed configuration) and 6B (open configuration). Referring to FIGS. 6A and 6B, the securement device 800 may include a base 810 and cuff 840a as described above. In the exemplary embodiment shown in FIGS. 6A and 6B the securement device 800 further includes a slide wing 815. The slide wing 815 comprises a moveable cuff 840b and a hole 825 configured to engage with a latch 838 when the securement device 600 is in the closed position, the latch 838 extending from the top face 820 of the base 810.

The slide wing 815 is coupled to the base 810 via a flexible joint 832. Exemplary flexible joints 832 can include hinges and living hinges. In some embodiments the flexible joint 832 need not be a hinge, but may be constructed of a flexible material. In some embodiments, flexible joint 832 can be flexible, particularly, relative to the base 810, cuff 840a, and slide wing 815, and the base 810, cuff 840a, and slide wing 815 can be rigid, relative to the flexible joint 832, even when the base 810, cuff 840a, slide wing 815, and flexible joint 832 are made of the same material.

The bar slide wing 815 is moveable between a closed position (shown in FIG. 6A) and an open position (shown in FIG. 6B). When the slide wing 815 is in the closed position, a portion of the slide wing 815 is positioned next to the entry gap 842 of the cuff 840a, such that slide wing 815 acts to block the entry gap 842, i.e., the slide wing 815 cooperates with the cuff 840a entry gap 842 in the closed position to form an obstacle to a medical article from leaving the interior aperture 843 after it has been positioned therein.

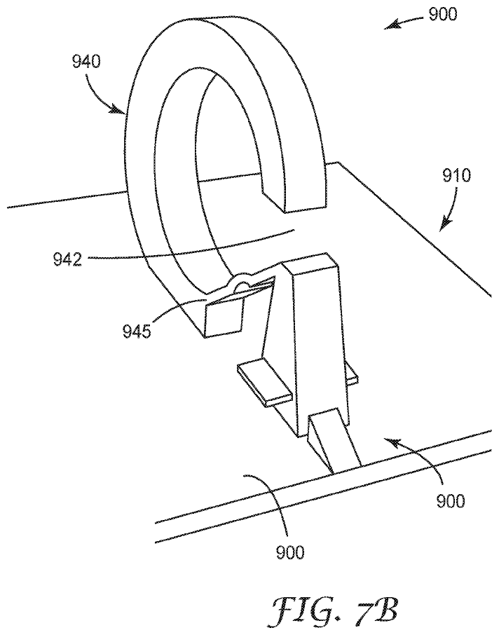

Another embodiment of a securement device 900 is shown in FIGS. 7A (open configuration) and 7B (closed configuration). Referring to FIGS. 7A and 7B, the securement device 900 includes a base 910 and cuff 940 substantially as described above, where the cuff 940 includes a cuff edge 945 coupled to a flexible arm 970 via a flexible joint 932. Exemplary flexible joints 932 can include hinges and living hinges. In some embodiments the flexible joint 932 need not be a hinge, but may be constructed of a flexible material. In some embodiments, flexible joint 932 can be flexible, particularly, relative to the base 910 and cuff 940, and base 910 and cuff 940 can be rigid, relative to the flexible joint 932, even when the base 910, cuff 940, and flexible joint 932 are made of the same material. Examples of flexible arms 970 suitable for use in embodiments of the present disclosure are described in U.S. Pub. No. 2015/0038912 (Karim et al.).

The securement device 900 further includes a stopper 971 extending from the top face 920 of the base 910, the flexible arm 970 configured to engage with the stopper 931. The flexible arm 970 cooperates with the cuff 940 in the closed position to partially block entry gap 942 (shown in FIG. 7B).

The securement devices 800, 900 can be used and applied substantially similarly to the securement device 100 described above. For example, the securement devices 800, 900 can be applied to a support device substantially similarly to the support device 200 described above to form securement systems.

### Select Embodiments of the Present Disclosure

The following embodiments are intended to be illustrative of the present disclosure and not limiting.

Embodiment 1 is a securement device for securing a medical article, comprising: a base having a longitudinal axis, a top face and a bottom face, wherein the longitudinal axis defines a longitudinal direction; a cuff disposed on the top face of the base and positioned along the longitudinal axis, the cuff comprising an entry gap in at least a portion of the cuff and defining an interior aperture; and a bar disposed on the top face of the base, the bar positioned along the longitudinal axis; wherein the bar is moveable between an open position and a closed position and wherein the bar cooperates with the cuff in the closed position to secure the medical article.

Embodiment 2 is a securement device according to embodiment 1, wherein the medical article is selected from the group consisting of a connector fitting, a catheter, a fluid supply line, and combinations thereof.

Embodiment 3 is a securement device according to embodiment 1 or embodiment 2, wherein the base has a rectangular shape.

Embodiment 4 is a securement device according to any one of embodiments 1-3, wherein the cuff is configured to receive and resiliently retain a generally cylindrical object.

Embodiment 5 is a securement device according to any one of embodiments 1-4, wherein the entry gap in the cuff is smaller than the interior aperture diameter defined by the cuff.

Embodiment 6 is a securement device according to any one of embodiments 1-5, wherein the length of the entry gap is less than 50%, less than 45%, less than 40%, less than 35%, less than 30%, less than 25%, or less than 20% of the interior aperture circumference.

Embodiment 7 is a securement device according to any one of embodiments 1-6, wherein the length of the entry gap is 5% to 45%, 10% to 45%, 15% to 40%, or 15% to 35% of the interior aperture circumference.

Embodiment 8 is a securement device according to any one of embodiments 1-7, wherein the cuff is generally annular in shape.

Embodiment 9 is a securement device according to any one of embodiments 1-8, wherein the interior aperture is generally circular in shape.

Embodiment 10 is a securement device according to any one of embodiments 1-9, wherein the cuff and the bar are each independently coupled to the base.

Embodiment 11 is a securement device according to any one of embodiments 1-10, wherein the bar comprises a post and a shaft and wherein the post is coupled to the shaft with a flexible joint.

Embodiment 12 is a securement device according to embodiment 11, wherein the shaft comprises a first shaft end and a second shaft end, wherein the first shaft end is coupled to the post with the flexible joint and wherein the second shaft end is positioned next to the entry gap.

Embodiment 13 is a securement device according to embodiment 12, wherein the second shaft end blocks up to 20%, up to 30%, up to 40%, up to 50%, up to 60%, up to 70%, up to 80%, up to 90%, or up to 100% of the entry gap.

Embodiment 14 is a securement device according to embodiment 12 or embodiment 13, wherein the second shaft end further comprises a compressible segment.

Embodiment 15 is a securement device according to embodiment 14, wherein the cuff further comprises a lower cuff edge and wherein the compressible segment is coupled to the lower cuff edge.

Embodiment 16 is a securement device according to embodiment 14 or embodiment 15, wherein the compressible segment is coupled to the base.

Embodiment 17 is a securement device according to any one of embodiments 1-16, the securement device further comprising a second cuff, the second cuff disposed on the top face of the base and spaced a distance from the first cuff along a line perpendicular to longitudinal axis, the second cuff comprising a second entry gap in at least a portion of the second cuff and defining a second interior aperture.

Embodiment 18 is a securement device according to embodiment 17, wherein the first entry gap and the second entry gap are aligned on the same side of the securement device.

Embodiment 19 is a securement device according to embodiment 17 or embodiment 18, wherein the first entry gap and the second entry gap are on opposite sides of the securement device.

Embodiment 20 is a securement system comprising: a securement device according to any one of claims 1-19; and a support device.

All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. A securement device for securing a medical article, comprising:
a base having a longitudinal axis, a top face and a bottom face, wherein the longitudinal axis defines a longitudinal direction;
a cuff disposed on the top face of the base and positioned along the longitudinal axis, the cuff comprising an entry gap in at least a portion of the cuff and defining an interior aperture; and
a bar disposed on the top face of the base, the bar positioned along the longitudinal axis;
wherein the bar is moveable between an open position and a closed position and wherein the bar cooperates with the cuff in the closed position to secure the medical article.

2. The securement device of claim 1, wherein the medical article is selected from the group consisting of a connector fitting, a catheter, a fluid supply line, and combinations thereof.

3. The securement device of claim 1, wherein the base has a rectangular shape.

4. The securement device of claim 1, wherein the cuff is configured to receive and resiliently retain a generally cylindrical object.

5. The securement device of claim 1, wherein the entry gap in the cuff is smaller than the interior aperture diameter defined by the cuff.

6. The securement device of claim 1, wherein the length of the entry gap is less than 50% of the interior aperture circumference.

7. The securement device of claim 1, wherein the length of the entry gap is 5% to 45% of the interior aperture circumference.

8. The securement device of claim 1, wherein the cuff is generally annular in shape.

9. The securement device of claim 1, wherein the interior aperture is generally circular in shape.

10. The securement device of claim 1, wherein the cuff and the bar are each independently coupled to the base.

11. The securement device of claim 1, wherein the bar comprises a post and a shaft and wherein the post is coupled to the shaft with a flexible joint.

12. The securement device of claim 11, wherein the shaft comprises a first shaft end and a second shaft end, wherein the first shaft end is coupled to the post with the flexible joint and wherein the second shaft end is positioned next to the entry gap.

13. The securement device of claim 12, wherein the second shaft end blocks up to 20% of the entry gap.

14. The securement device of claim 12, wherein the second shaft end further comprises a compressible segment.

15. The securement device of claim 14, wherein the cuff further comprises a lower cuff edge and wherein the compressible segment is coupled to the lower cuff edge.

16. The securement device of claim 14, wherein the compressible segment is coupled to the base.

17. The securement device of claim 1, further comprising a second cuff, the second cuff disposed on the top face of the base and spaced a distance from the first cuff along a line perpendicular to longitudinal axis, the second cuff comprising a second entry gap in at least a portion of the second cuff and defining a second interior aperture.

18. The securement device of claim 17, wherein the first entry gap and the second entry gap are aligned on the same side of the securement device.

19. The securement device of claim 17, wherein the first entry gap and the second entry gap are on opposite sides of the securement device.

20. A securement system comprising:
a securement device of claim 1; and
a support device.
